(19) United States
(12) Patent Application Publication
(10) Pub. No.: US 2024/0226429 A9
(43) Pub. Date: Jul. 11, 2024

(54) SELF-INFLATING CUFF FOR USE WITH IV SET

(71) Applicant: CareFusion 303, Inc., San Diego, CA (US)

(72) Inventor: Nolan Leendert Pupping, Carlsbad, CA (US)

(21) Appl. No.: 18/493,432

(22) Filed: Oct. 24, 2023

Related U.S. Application Data
(60) Provisional application No. 63/418,900, filed on Oct. 24, 2022.

(51) Int. Cl. A61M 5/148

(52) U.S. Cl. CPC A61M 5/1483; A61M 2205/0216; A61M 2205/3331; A61M 2206/20

(57) ABSTRACT

A self-inflating cuff and methods of using the self-inflating cuff are provided. The self-inflating cuff includes first and second compartments formed within the self-inflating cuff and housing respective substances. The first and second compartments are configured to rupture in response to the force applied to an exterior surface of the self-inflating cuff, which causes the respective substances to combine to initiate a chemical reaction to produce a reactionary force on an interior surface of the self-inflating cuff that causes the self-inflating cuff to expand. The self-inflating cuff is further configured to expand such that a first opening at a first end of the self-inflating cuff and a second opening at a second end opposite the first end of the self-inflating cuff enlarge enough to receive a container through the first opening and a tubing of the container through the second opening.

### CROSS REFERENCE TO RELATED APPLICATION

This application claims the benefit of priority under 35 U.S.C. § 119 to U.S. Provisional Patent Application Ser. No. 63/418,900, entitled “SELF-INFLATING CUFF FOR USE WITH IV SET”, filed on Oct. 24, 2022, the disclosure of which is incorporated herein by reference in its entirety.

### FIELD OF THE INVENTION

The present disclosure generally relates to medical fluid distribution systems, and, in particular, to self-inflating cuff for distributing a medical fluid from an IV bag.

### BACKGROUND

Medical treatments often include the infusion of a medical fluid (e.g., a saline solution or a liquid medication) to patients using an infusion pump that distributes the medical fluid from an intravenous (IV) bag to a patient. When an infusion pump is unavailable, medical practitioners use their hands to compress or otherwise squeeze on the IV bag to administer the medical fluid. Manually compressing the IV bag can result in hand fatigue which greatly inconveniences medical practitioners.

As such, there is a need for devices and methods of use thereof that can distribute medical fluids from an intravenous (IV) bag to a patient without fatiguing a medical practitioner.

### SUMMARY

The disclosed subject matter relates to self-inflating cuff for use with IV bags. In some implementations, the self-inflating cuff includes an exterior surface and an interior surface (e.g., an inflatable body). The interior surface includes a first membrane layer formed within and along the interior surface at a first position, and a second membrane layer formed within and along the interior surface at a second position. The first membrane layer forms a first compartment that is configured to house a first and the second membrane layer forms a second compartment, separate from the first compartment, that is configured to house a second substance in isolation from the first substance. The first membrane layer is configured to rupture in response to a force applied to the exterior surface of the self-inflating cuff, and the second membrane layer is also configured to rupture in response to the force applied to the exterior surface of the self-inflating cuff. The self-inflating cuff is configured to, when the first and second compartments rupture in response to the force applied to the exterior surface, cause the first and second substances to combine to initiate a chemical reaction to produce a reactionary force on the interior surface of the self-inflating cuff that causes the self-inflating cuff to expand. The self-inflating cuff is further configured to expand such that a first opening at a first end of the self-inflating cuff and a second opening at a second end opposite the first end of the self-inflating cuff enlarge enough to receive a container through the first opening and a tubing of the container through the second opening. In some implementations, after the container and the tubing of the container are received via the first and second ends of the self-inflating cuff, respectively, and the reactionary force applied to the interior surface of the self-inflating cuff is removed at completion of the chemical reaction, the self-inflating cuff is configured to self-contract and compress the container such that a fluid flowing from the container, when flowing, is accelerated.

In some implementations, the first compartment is adjacent to the second compartment within the self-inflating cuff. Alternatively, in some implementations, the first compartment is formed within a first portion of the self-inflating cuff adjacent to the first end, and the second compartment is formed within a second portion of the self-inflating cuff opposite the first portion at the second end, and vice versa.

In some implementations, the force applied to the exterior surface of the self-inflating cuff is a compression force applied to the first and second ends of the self-inflating cuff. Alternatively in some implementations, the force applied to the exterior surface of the self-inflating cuff is a tension force applied to the first and second ends of the self-inflating cuff. In some implementations, the first compartment is configured to rupture before the second compartment when the force is applied to the exterior surface of the self-inflating cuff. In some implementations, the first compartment includes a first predetermined amount of the first substance and the second compartment includes a second predetermined amount of the second substance, the first predetermined amount of the first substance and the second predetermined amount of the second substance are based on a volume of the self-inflating cuff. In some implementations, the self-inflating cuff includes a resilient material such that, after the self-inflating cuff expands, the self-inflating cuff self-contracts at a predetermined rate until the self-inflating cuff returns to an unexpanded state.

In another aspect, a method of inflating a self-inflating cuff is disclosed. The method includes providing a self-inflating cuff having a first membrane layer formed within and along the interior surface at a first position and a second membrane layer formed within and along the interior surface at a second position. The first membrane layer forms a first compartment that is configured to houses a first substance and the second membrane layer forms a second compartment, separate from the first compartment, that is configured to houses a second substance in isolation from the first substance. The method includes applying a force to the exterior surface of the self-inflating cuff, the force causing the first membrane layer and the second membrane layers to rupture allowing the first and second substances to combine. The method also includes initiating a chemical reaction based on a combination of the first and second substances to produce a reactionary force on the interior surface of the self-inflating cuff that causes the self-inflating cuff to expand such that a first opening at a first end of the self-inflating cuff and a second opening at a second end opposite the first end of the self-inflating cuff enlarge enough to receive a container through the first opening and a tubing of the container through the second opening. The method further includes receiving the container and the tubing of the container via the first and second ends of the self-inflating cuff. In some embodiments, the method includes after the container and the tubing of the container are received via the first and second ends of the self-inflating cuff, respectively, and the reactionary force applied to the interior surface of the self-inflating cuff is removed at completion of the chemical reaction, initiating self-contraction of the self-inflating cuff and compression of the container such that a fluid flowing from the container, when flowing, is accelerated

In some implementations, the self-inflating cuff includes a resilient material such that, after the self-inflating cuff expands, the self-inflating cuff self-contracts at a predetermined rate until the self-inflating cuff returns to an unexpanded state. In some implementations, the force applied to the exterior surface of the self-inflating cuff is a compression force applied to the first and second ends of the self-inflating cuff. Alternatively, in some implementations, the force applied to the exterior surface of the self-inflating cuff is a tension force applied to the first and second ends of the self-inflating cuff.

In some implementations, the first compartment is adjacent to the second compartment within the self-inflating cuff. Alternatively, in some implementations, the first compartment is formed within a first portion adjacent to the first end of the self-inflating cuff and the second compartment is formed within a second portion of the self-inflating cuff opposite the first portion and adjacent to the second end, and vice versa. In some implementations, the first compartment is configured to rupture before the second compartment when the force is applied to the exterior surface of the self-inflating cuff. In some implementations, the first compartment includes a first predetermined amount of the first substance and the second compartment includes a second predetermined amount of the second substance. The first predetermined amount of the first substance and the second predetermined amount of the second substance are based on a volume of the self-inflating cuff.

Note that the various implementations described above can be combined with other implementations described herein (e.g., keep an impedance for a single or group of neuromuscular-signal sensors can be combined with matching of impedances, such that impedances can be both matched and be kept within particular ranges of impedance values). The features and advantages described in the specification are not all inclusive and, in particular, additional features and advantages will be apparent to one of ordinary skill in the art in view of the drawings, specification, and claims. Moreover, it should be noted that the language used in the specification has been principally selected for readability and instructional purposes.

### BRIEF DESCRIPTION OF THE DRAWINGS

So that the present disclosure can be understood in greater detail, a more particular description may be had by reference to the features of various implementations, some of which are illustrated in the appended drawings. The appended drawings, however, merely illustrate pertinent features of the present disclosure. The description may admit to other effective features as the person of skill in this art will appreciate upon reading this disclosure.

FIG. 1 illustrates an IV pump administering a medical fluid to a patient.

FIGS. 2A-2D illustrates a self-inflating cuff, in accordance with some implementations.

FIG. 3 is flowcharts illustrating a method 300 of accelerating a flow of fluid from a container, in accordance with some implementations.

FIG. 4 is a conceptual diagram illustrating an example electronic system for controlling a pump, according to aspects of the subject technology.

In accordance with common practice, the various features illustrated in the drawings are not drawn to scale. Accordingly, the dimensions of the various features may be arbitrarily expanded or reduced for clarity. In addition, some of the drawings may not depict all of the components of a given system, method, or device. Finally, like reference numerals denote like features throughout the specification and figures.

### DETAILED DESCRIPTION

Numerous details are described herein in order to provide a thorough understanding of the example implementations illustrated in the accompanying drawings. However, some implementations may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the implementations described herein.

The disclosed self-inflating cuff is configured to receive an IV bag and accelerate a medical fluid from the IV bag to a patient for treatment. In particular, the self-inflating cuff is configured to self-expand responsive to a force applied to a body of the self-inflating cuff. After the force is removed from the body of the self-inflating cuff and the body of the self-inflating stretches to an expanded state. In some implementations, the self-inflating cuff self-expands due to a chemical reaction caused by at least two substances housed within the self-inflating cuff that are caused to mix in response to the force applied to the self-inflating cuff. The self-inflating cuff is further configured to receive the IV bag and self-contract compressing the IV bag and accelerating a flow of fluid from the IV bag to a patient.

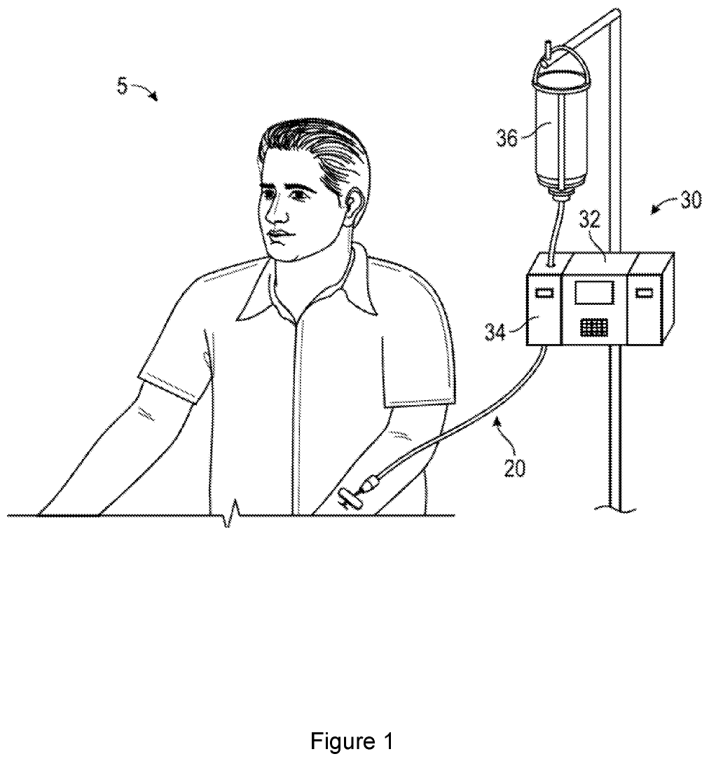

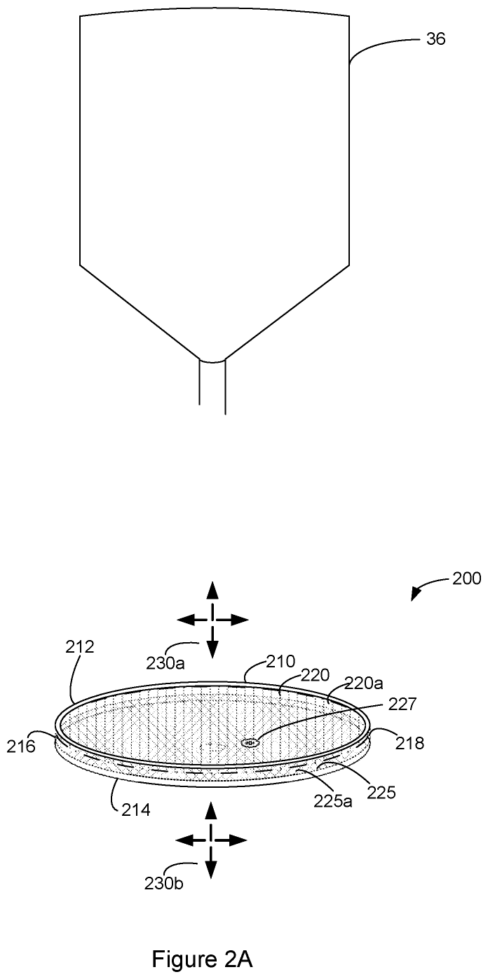

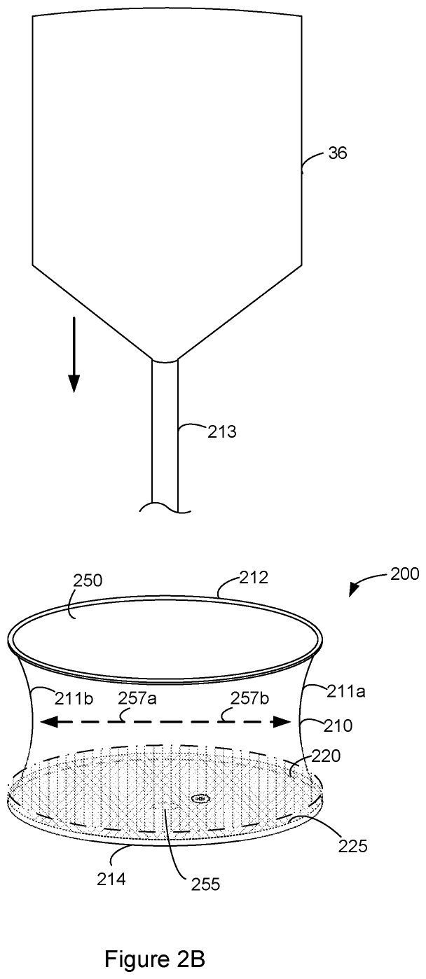

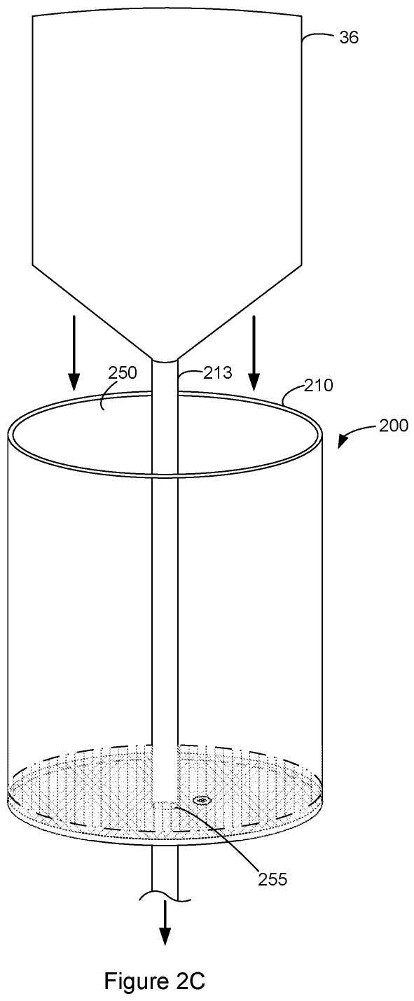

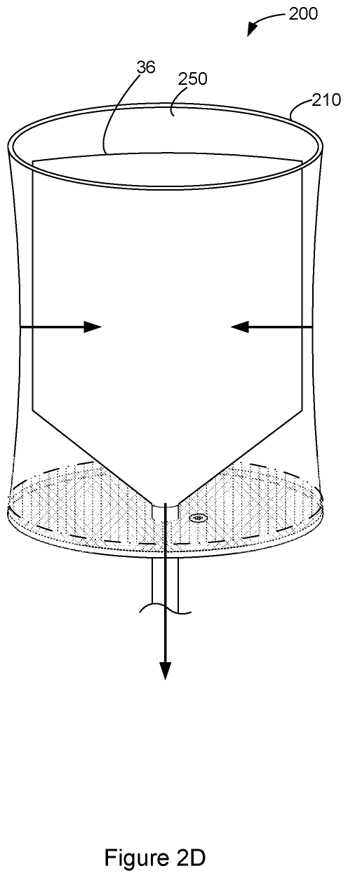

FIG. 1 illustrates an IV pump administering a medical fluid to a patient. The IV pump 30 includes a controller 32 and one or more pump modules 34. An IV set 20 is connected between a container 36 (e.g., an IV bag) of the medical fluid and the patient 5. During operation, IV pump 30 delivers the medical fluid to the patient 5. The IV pump 30 is configured to administer the medical fluid to the patient at a predetermined and uniform rate. When an IV pump 30 is unavailable or otherwise out of service, the medical fluid can be distributed to the patient 5 by applying a force to the container 36 (e.g., squeezing the container 36). In some implementations, a self-inflating cuff 200 (FIGS. 2A-2D) is used to administer the medical fluid to the patient 5 in place of the IV pump 30, as discussed below.

FIGS. 2A-2D illustrate a self-inflating cuff, in accordance with some implementations. The self-inflating cuff 200 includes an inflatable body 210, a first compartment 220, and a second compartment 225. The first compartment 220 is formed within and along the interior surface of the inflatable body 210 and is configured to house a first substance. More specifically, in some embodiments, the first compartment 220 is formed by a first membrane layer 220a (represented by a vertical fill pattern) formed within and along the interior surface at a first position. The second compartment 225 is formed within and along the inflatable body 210 and is configured to house a second substance in isolation from the first substance. Similar to the first compartment 220, in some embodiments, the second compartment 225 is formed by a second membrane layer 225a (represented by a cross fill pattern) formed along and within the interior surface at a second position. As described in detail below, the self-inflating cuff 200, is configured to, when the first and second compartments 220 and 225 rupture in response to the force applied to the exterior surface, cause the first and second substances to combine to initiate a chemical reaction to produce a reactionary force on the interior surface of the self-inflating cuff that causes the self-inflating cuff to expand. The self-inflating cuff 200 is further configured to expand such that a first opening at a first end of the self-inflating cuff and a second opening at a second end opposite the first end of the self-inflating cuff 200 enlarge enough to receive a container 36 through the first opening and a tubing of the container through the second opening.

In some implementations, the first compartment 220 is adjacent to the second compartment 225 within the inflatable body 210. For example, the first compartment 220 can be disposed above the second compartment 225 or vice versa. As described above, in some implementations, the first compartment 220 is formed at a first portion of the inflatable body 210 (e.g., by a breakable first membrane layer 220a that holds the first substance in isolation) and the second compartment 225 is formed at a second portion of the inflatable body 210 opposite the first portion (e.g., by a breakable second membrane layer 220b that holds the second substance in isolation). For example, the first compartment 220 can be formed near the first end 212 and the second compartment 225 can be formed near the second end 214. In some implementations, the first and second membrane layers include one of a include foil, plastic, paper and/or other rupturable or breakable material.

In some implementations, the first compartment 220 includes a first predetermined amount of the first substance and the second compartment 225 includes a second predetermined amount of the second substance. The first and second substances, when mixed (combined or when they come in contact with one another), cause a chemical reaction that generates at least a gas. The generated gas expands and applies a reactionary force 257 (FIG. 2B) to the interior surface of the inflatable body 210. More specifically, the chemical reaction caused by the first and second substances generates a reactionary force 257 that expands the inflatable body 210. In some implementations, the first predetermined amount of the first substance and the second predetermined amount of the second substance are based on a volume of the inflatable body. The first substance and second substances can be at least any two substances that, when mixed, generate non-harmful gasses. For example, the first substance can be vinegar and the second substance can be baking soda.

The first and second substances are released from the first and second compartments 220 and 225 responsive to application of a force 230 on the exterior surface 211a (FIG. 2B) of the inflatable body 210. In particular, the first and second membrane layers 220a and 225a rupture 227 such that the first and second substances combine causing a chemical reaction. As shown in FIG. 2B, the chemical reaction generates a reactionary force 257 applied to the interior surface 211b (FIG. 2B) of the inflatable body 210 that causes the inflatable body 210 to expand such that at least a first opening 250 (FIG. 2B) at a first end 212 of the inflatable body 210 and a second opening 255 (FIG. 2B) at a second end 214 opposite the first end 212 of the inflatable body 210 enlarge. The first end 212 is configured to receive a container and the second end is configured to receive a tubing of the container. In other words, the self-inflating cuff 200 self-expands responsive to the force 230 applied to inflatable body 210. In some implementations, the first compartment 220 is configured to brake before the second compartment 225 when the force 230 is applied to the inflatable body 210 or vice versa. Alternatively, in some implementations, the first compartment and the second compartment are configured to break at the same time.

In some embodiments, the first and second openings 250 and 255 are sealed until the force 230 is applied to the exterior surface 211a of the inflatable body 210 and the first and second membrane layers 220a and 225a rupture. The first and second openings 250 and 255 remain substantially small (or sealed) until the inflatable body 210 reaches a predetermined size such that the first and second substances do not escape the inflatable body 210 and/or the chemical reaction is ended before completion. Substantially small, in some implementations, means that the first and second openings are air or fluid cannot escape via the first and second openings 250 and 255. In some embodiments, the predetermined size is at least three fourths (¾) of the total size of the inflatable body 210. Alternatively, in some embodiments, the self-inflating cuff 200 is packaged in a sealed container that, responsive to the force 230 being applied to the sealed container, cause the inflatable body to expand and, once expanded, the sealed container can be broken and the self-inflating cuff 200 removed from the sealed container.

The force 230 (e.g., to break a compartment(s)) can be a compression force (e.g., forces 230a and 230b) applied to a first end 212 and a second end 214 (opposite the first end 212) of the inflatable body 210. For example, a medical practitioner can compress (e.g., squeeze) the first end 212 and the second end 214 of the inflatable body 210 between their hands to apply compression forces 230a and 230b, press down on the first end 212 or the second end 214 of the inflatable body 210 against a surface (e.g., a table, a wall, etc.), etc. Alternatively, in some implementations, the force 230 is a tension force (represented by multidirectional arrows 230a and 230b) applied to a first end 212 and a second end 214 of the inflatable body 210. For example, a medical practitioner can pull the first end 212 and the second end 214 of the inflatable body 210 in opposite directions. In some embodiments, the force 230 is a lateral force can be applied to the inflatable body 210 the first end 212 and the second end 214 of the inflatable body 210. Alternatively, in some implementations, the force 230 is applied to the sides 216 and 218 of the inflatable body 210. The above examples are non-limiting, any force that ruptures the first and second membrane layers 220a and 225a can be used.

Turning to FIG. 2B, the inflatable body 210 of the self-inflating cuff 200 expands as a result of the chemical reaction caused by the first and second substances. More specifically, the chemical reaction caused by the first and second substances generate one or more reaction forces 257a and 257b (e.g., a pressure force) applied to interior surface 211b of the inflatable body 210 that cause the inflatable body 210 to expand such that the inflatable body 210 can receive the container 36 and tubing 213 of the container 36. Although not shown, the reaction forces 257 are applied in all directions such that the inflatable body expands at a faster rate. The reaction forces 257 are applied to the interior surface 211b of the inflatable body 210 until the chemical reaction between the first and second substances completes. The first end 212 of the inflatable body 210 includes a first opening 250 configured to receive the container 36 (when the inflatable body 210 is expanded) and the second end 214 of the inflatable body 210 includes a second opening 255 configured to receive tubing 213 of the container 36 (as shown in FIG. 2C). In particular, as the inflatable body 210 expands, the first opening 250 at the first end 212 of the inflatable body 210 and the second opening 255 at the second end 214 opposite the first end 212 of the inflatable body 210 enlarge such that the first end can receive the container 36 and the second end can receive the tubing 213 of the container 36.

FIG. 2C shows the self-inflating cuff 200 with the inflatable body 210 substantially expanded. As described above in reference to FIG. 2B, after a force 230 is applied to the exterior surface 211a of the inflatable body 210 that causes the first and second membrane layers 220a and 225b to rupture, one or more reaction forces 257a and 257b (generated by the chemical reaction of the first and second substances when combined) applied to an interior surface 211b of the inflatable body 210 cause the inflatable body 210 to expand such that the inflatable body 210 can receive the container 36 and tubing 213 of the container 36. The inflatable body 210 continues to expand until the chemical reaction between the first and second substances completes. Once substantially expanded, the container 36 can be placed within the inflatable body 210 of the self-inflating cuff 200. Substantially expanded means, in some implementations, that the inflatable body 210 is expanded such that it can receive at least 70 percent of the container 36. As described above in reference to FIG. 2B, after the inflatable body 210 is expanded, the tubing 213 of the container 36 can be received by the second opening 255 of the inflatable body 210. The second opening 255 of the inflatable body 210 does not obstruct or occlude the flow of medical fluid from the container 36 (to a patient). By allowing the tubing 213 of the container 36 to be received the second opening 255 without interfering with the flow of medical fluid, the self-inflating cuff 200 is able to administer the medical fluid from the container 36 to the patient efficiently without impeding the flow of medical fluid.

FIG. 2D illustrates the container placed within the inflatable body 210, via the first opening 250, and held within the inflatable body 210 as it is compressed by self-contraction of the inflatable body 210. As the inflatable body 210 contracts, medical fluid flowing from the container 36, when allowed to flow, is accelerated from the container 36 (i.e., medical fluid flows from the container 36 to a patient). In some implementations, the inflatable body 210 self-contracts once the reactionary forces 257 are removed from the interior surface 211b of the inflatable body 210. In some implementations, the inflatable body 210 is formed of a resilient material such that, after the inflatable body 210 expands (e.g., while the chemical reaction between at least the first and second substances is ongoing) and self-contracts (e.g., after the chemical reaction between at least the first and second substances ends (i.e., stop generating a non-toxic gas that causes the inflatable body 210 to expand) at a predetermined rate until the inflatable body 210 returns to an unexpanded state. The predetermined rate at which the inflatable body 210 self-contracts is based, at least in part, on the resilient material. In some implementations, the resilient material includes rubber, latex, silicone, polyurethane, nylon, thermoplastic elastomers, and/or other flexible material that expands and self-contracts based on the application of a force.

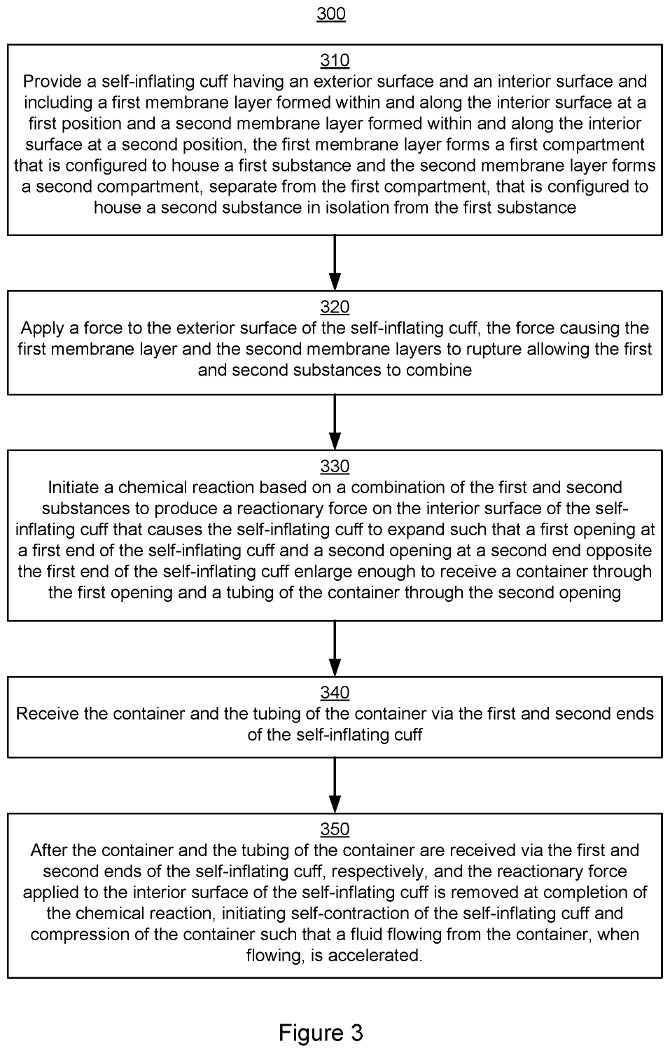

FIG. 3 is flowcharts illustrating a method 300 of accelerating a flow of fluid from a container, in accordance with some implementations. Method 300 can be performed by a self-inflating cuff 200 described above in reference to FIGS. 2A-2D. Methods consistent with the present disclosure may include at least some, but not all, of the operations illustrated in method 300, performed in a different sequence. Furthermore, methods consistent with the present disclosure may include at least two or more steps as in method 300 performed overlapping in time, or almost simultaneously. The method 300 includes providing (310) a self-inflating cuff having an exterior surface and an interior surface and including a first membrane layer formed within and along the interior surface at a first position and a second membrane layer formed within and along the interior surface at a second position. The first membrane layer forms a first compartment that is configured to house a first substance and the second membrane layer forms a second compartment, separate from the first compartment, that is configured to house a second substance in isolation from the first substance.

Method 300 includes applying (320) a force to the exterior surface of the self-inflating cuff, the force causing the first membrane layer and the second membrane layers to rupture allowing the first and second substances to combine. For example, as described above in reference to FIGS. 2A-2C, a force applied to a portion of inflatable body 210 of the self-inflating cuff 200 causes the first and second compartments 220 and 225 to break. In some implementations, the first compartment is configured to brake before the second compartment or vice versa. Alternatively, in some implementations, first and second compartments are configured to break at the same time. In some implementations, the force applied to the inflatable body is a compression or tension force. In some implementations, the first compartment is adjacent to the second compartment within the inflatable body.

The method includes, initiating (330) a chemical reaction based on a combination of the first and second substances (which are allowed to combine when the force is applied on the exterior surface of the inflatable body and the first and second compartments rupture) to produce a reactionary force on the interior surface of the self-inflating cuff that causes the self-inflating cuff to expand such that a first opening at a first end of the self-inflating cuff and a second opening at a second end opposite the first end of the self-inflating cuff enlarge enough to receive a container (e.g., an IV bag) through the first opening and a tubing of the container (e.g., tubing 213 of an IV bag) through the second opening. In some implementation, the first and second openings are sealed until the first and second membrane layers are ruptured. Once the force is applied on the exterior surface of the inflatable body, the first and second openings remain substantially small until the inflatable body reaches a predetermined size such that the chemical reaction is not cut short. In some embodiments, the predetermined size is at least three fourths (¾) of the total size of the inflatable body 210.

In some embodiments, the inflatable body is formed of a resilient material such that, after the inflatable body expands, the inflatable body self-contracts at a predetermined rate until the inflatable body returns to an unexpanded state. In some implementations, the first compartment includes a first predetermined amount of the first substance and the second compartment includes a second predetermined amount of the second substance. The first predetermined amount of the first substance and the second predetermined amount of the second substance are based on a volume of the inflatable body. Additional information on the first and second substances is provided above in reference to FIGS. 2A-2D.

The method 300 includes receiving (340) the container and the tubing of the container via the first and second ends of the inflatable body and, after the container and the tubing of the container are received via the first and second ends of the self-inflating cuff, respectively, and the reactionary force applied to the interior surface of the self-inflating cuff is removed at completion of the chemical reaction, initiating (350) self-contraction of the self-inflating cuff and compression of the container such that a fluid flowing from the container, when flowing, is accelerated.

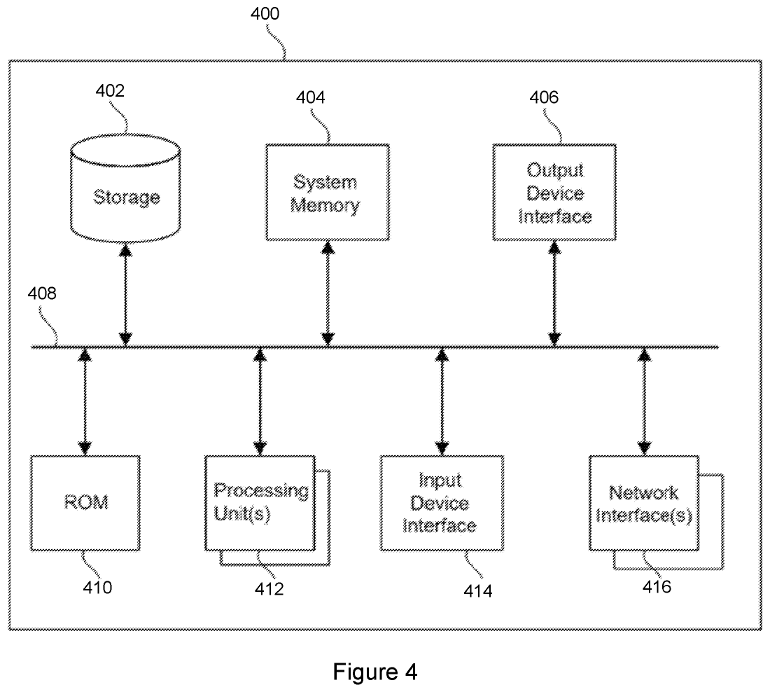

FIG. 4 is a conceptual diagram illustrating an example electronic system 400 for controlling a pump, according to aspects of the subject technology. Electronic system 400 may be a specifically configured computing device for execution of software associated with components and processes provided by FIGS. 1 through 5, including but not limited to controller 32 of IV pump 30. Electronic system 400 may be representative, in combination with the disclosure regarding FIGS. 1 through 3.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 414, an output device interface 406, and one or more network interfaces 416. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process, in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such as a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations. Such storage and/or memory devices 402, 404 may be representative of memory of controller 32.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”), such as those shown in controller 32 of FIG. 1. Output device interfaces 406 (e.g., shown as a display in the controller 60 of FIG. 1) enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML, page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A self-inflating cuff, comprising: an exterior surface; and an interior surface. The interior surface including a first membrane layer formed within and along the interior surface at a first position, the first membrane layer forms a first compartment that is configured to house a first substance and to rupture in response to a force applied to the exterior surface of a self-inflating cuff. The interior surface also includes a second membrane layer formed within and along the interior surface at a second position, wherein the second membrane layer forms a second compartment, separate from the first compartment, that is configured to house a second substance in isolation from the first substance and to rupture in response to the force applied to the exterior surface of the self-inflating cuff. The self-inflating cuff is configured to, when the first and second compartments rupture in response to the force applied to the exterior surface, cause the first and second substances to combine to initiate a chemical reaction to produce a reactionary force on the interior surface of the self-inflating cuff that causes the self-inflating cuff to expand; and the self-inflating cuff is further configured to expand such that a first opening at a first end of the self-inflating cuff and a second opening at a second end opposite the first end of the self-inflating cuff enlarge enough to receive a container through the first opening and a tubing of the container through the second opening.

Clause 2. The self-inflating cuff of Clause 1, the self-inflating cuff is configured to, after the container and the tubing of the container are received via the first and second ends of the self-inflating cuff, respectively, and the reactionary force applied to the interior surface of the self-inflating cuff is removed at completion of the chemical reaction, self-contract and compress the container such that a fluid flowing from the container, when flowing, is accelerated.

Clause 3. The self-inflating cuff of Clause 1 or Clause 2, the first compartment is adjacent to the second compartment within the self-inflating cuff.

Clause 4. The self-inflating cuff of any of Clause 1 through Clause 3, the force applied to the exterior surface of the self-inflating cuff is a compression force applied to the first and second ends of the self-inflating cuff.

Clause 5. The self-inflating cuff of any of Clause 1 through Clause 3, the force applied to the exterior surface of the self-inflating cuff is a tension force applied to the first and second ends of the self-inflating cuff.

Clause 6. The self-inflating cuff of any of Clause 1 through Clause 5, the first compartment is configured to rupture before the second compartment when the force is applied to the exterior surface of the self-inflating cuff.

Clause 7. The self-inflating cuff of any of Clause 1 through Clause 6, the first compartment includes a first predetermined amount of the first substance and the second compartment includes a second predetermined amount of the second substance, and the first predetermined amount of the first substance and the second predetermined amount of the second substance are based on a volume of the self-inflating cuff.

Clause 8. The self-inflating cuff of any of Clause 1 through Clause 7, the self-inflating cuff comprise a resilient material such that, after the self-inflating cuff expands, the self-inflating cuff self-contracts at a predetermined rate until the self-inflating cuff returns to an unexpanded state.

Clause 9. A method of inflating a self-inflating cuff, the method including providing a self-inflating cuff having an exterior surface and an interior surface and including a first membrane layer formed within and along the interior surface at a first position, the first membrane layer forming a first compartment that is configured to house a first substance; and a second membrane layer formed within and along the interior surface at a second position, the second membrane layer forming a second compartment, separate from the first compartment, that is configured to house a second substance in isolation from the first substance. The method further includes applying a force to the exterior surface of the self-inflating cuff, the force causing the first membrane layer and the second membrane layers to rupture allowing the first and second substances to combine initiating a chemical reaction based on a combination of the first and second substances to produce a reactionary force on the interior surface of the self-inflating cuff that causes the self-inflating cuff to expand such that a first opening at a first end of the self-inflating cuff and a second opening at a second end opposite the first end of the self-inflating cuff enlarge enough to receive a container through the first opening and a tubing of the container through the second opening. The method also includes receiving the container and the tubing of the container via the first and second ends of the self-inflating cuff.

Clause 10. The method of Clause 9, the method further including after the container and the tubing of the container are received via the first and second ends of the self-inflating cuff, respectively, and the reactionary force applied to the interior surface of the self-inflating cuff is removed at completion of the chemical reaction, initiating self-contraction of the self-inflating cuff and compression of the container such that a fluid flowing from the container, when flowing, is accelerated.

Clause 11. The method of any of Clause 9 and Clause 10, the force applied to the self-inflating cuff is a compression force applied to the exterior surface of the first and second ends of the self-inflating cuff.

Clause 12. The method of any of Clause 9 and Clause 10, the force applied to the self-inflating cuff is a tension force applied to the exterior surface of the first and second ends of the self-inflating cuff.

Clause 13. The method of any of Clause 9 through Clause 12, the first compartment is adjacent to the second compartment within the self-inflating cuff.

Clause 14. The method of any of Clause 9 through Clause 13, the first compartment is configured to rupture before the second compartment when the force is applied to the self-inflating cuff.

Clause 15. The method of any of Clause 9 through Clause 14, the first compartment includes a first predetermined amount of the first substance and the second compartment includes a second predetermined amount of the second substance, wherein the first predetermined amount of the first substance and the second predetermined amount of the second substance are based on a volume of the self-inflating cuff.

Clause 16. The method of any of Clause 9 through Clause 15, the self-inflating cuff comprises a resilient material such that, after the self-inflating cuff expands, the self-inflating cuff self-contracts at a predetermined rate until the self-inflating cuff returns to an unexpanded state.

Clause 17. An infusion system including a fluid source (e.g., a container including a fluid), tubing coupled with the fluid source, and a self-inflating cuff. The self-inflating cuff is configured to, responsive to a force applied to an exterior surface of the self-inflating cuff, i) initiate a chemical reaction caused by a first substance and a second substance within distinct compartments of the self-inflating cuff that are combined in response to the force, and ii) produce a reactionary force on an interior surface of the self-inflating cuff that causes the self-inflating cuff to expand. The self-inflating cuff is further configured to expands such that the self-inflating cuff is configured to i) receive and couple with the fluid source and ii) pass the tubing through an opening of the self-inflating cuff such that the self-inflating cuff does not occlude the tubing.

Clause 18. The infusion system of clause 18, the self-inflating cuff is configured to, after the reactionary force applied to the self-inflating cuff is removed at completion of the chemical reaction, self-contract and compress the fluid source such that a fluid flowing from the fluid source, when flowing, is accelerated.

Clause 19. The infusion system of Clause 17 or Clause 18, wherein the force applied to the exterior surface of the self-inflating cuff is a compression force.

Clause 20. The infusion system of Clause 17 or Clause 18, wherein the force applied to the exterior surface of the self-inflating cuff is a tension force.

Clause 21. The infusion system of any of Clauses 17 through 20, the self-inflating cuff is configured in accordance with the self-inflating cuff of any of Clause 1 through Clause 8.

Further Consideration:

In some implementations, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

It will be understood that, although the terms “first,” “second,” etc. may be used herein to describe various elements, these elements should not be limited by these terms. These terms are only used to distinguish one element from another. Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

The terminology used herein is for the purpose of describing particular implementations only and is not intended to be limiting of the claims. As used in the description of the implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all implementations, or one or more implementations. An embodiment may provide one or more examples. A phrase such an embodiment may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples, and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A self-inflating cuff, comprising:
an exterior surface; and
an interior surface including:
a first membrane layer formed within and along the interior surface at a first position, wherein the first membrane layer forms a first compartment that is configured to house a first substance and to rupture in response to a force applied to the exterior surface of a self-inflating cuff, and
a second membrane layer formed within and along the interior surface at a second position, wherein the second membrane layer forms a second compartment, separate from the first compartment, that is configured to house a second substance in isolation from the first substance and to rupture in response to the force applied to the exterior surface of the self-inflating cuff;
wherein the self-inflating cuff is configured to, when the first and second compartments rupture in response to the force applied to the exterior surface, cause the first and second substances to combine to initiate a chemical reaction to produce a reactionary force on the interior surface of the self-inflating cuff that causes the self-inflating cuff to expand; and
wherein the self-inflating cuff is further configured to expand such that a first opening at a first end of the self-inflating cuff and a second opening at a second end opposite the first end of the self-inflating cuff enlarge enough to receive a container through the first opening and a tubing of the container through the second opening.

2. The self-inflating cuff of claim 1, wherein the self-inflating cuff is configured to, after the container and the tubing of the container are received via the first and second ends of the self-inflating cuff, respectively, and the reactionary force applied to the interior surface of the self-inflating cuff is removed at completion of the chemical reaction, self-contract and compress the container such that a fluid flowing from the container, when flowing, is accelerated.

3. The self-inflating cuff of claim 1, wherein the first compartment is adjacent to the second compartment within the self-inflating cuff.

4. The self-inflating cuff of claim 1, wherein the force applied to the exterior surface of the self-inflating cuff is a compression force applied to the first and second ends of the self-inflating cuff.

5. The self-inflating cuff of claim 1, wherein the force applied to the exterior surface of the self-inflating cuff is a tension force applied to the first and second ends of the self-inflating cuff.

6. The self-inflating cuff of claim 1, wherein the first compartment is configured to rupture before the second compartment when the force is applied to the exterior surface of the self-inflating cuff.

7. The self-inflating cuff of claim 1, wherein:
the first compartment includes a first predetermined amount of the first substance and the second compartment includes a second predetermined amount of the second substance, and
the first predetermined amount of the first substance and the second predetermined amount of the second substance are based on a volume of the self-inflating cuff.

8. The self-inflating cuff of claim 1, wherein the self-inflating cuff comprise a resilient material such that, after the self-inflating cuff expands, the self-inflating cuff self-contracts at a predetermined rate until the self-inflating cuff returns to an unexpanded state.

9. A method of inflating a self-inflating cuff, comprising:
providing a self-inflating cuff having an exterior surface and an interior surface and including a first membrane layer formed within and along the interior surface at a first position, wherein the first membrane layer forms a first compartment that is configured to house a first substance, and a second membrane layer formed within and along the interior surface at a second position, wherein the second membrane layer forms a second compartment, separate from the first compartment, that is configured to house a second substance in isolation from the first sub stance;
applying a force to the exterior surface of the self-inflating cuff, wherein the force causes the first membrane layer and the second membrane layers to rupture allowing the first and second substances to combine initiating a chemical reaction based on a combination of the first and second substances to produce a reactionary force on the interior surface of the self-inflating cuff that causes the self-inflating cuff to expand such that a first opening at a first end of the self-inflating cuff and a second opening at a second end opposite the first end of the self-inflating cuff enlarge enough to receive a container through the first opening and a tubing of the container through the second opening; and
receiving the container and the tubing of the container via the first and second ends of the self-inflating cuff.

10. The method of claim 9, further comprising:
after the container and the tubing of the container are received via the first and second ends of the self-inflating cuff, respectively, and the reactionary force applied to the interior surface of the self-inflating cuff is removed at completion of the chemical reaction, initiating self-contraction of the self-inflating cuff and compression of the container such that a fluid flowing from the container, when flowing, is accelerated.

11. The method of claim 9, wherein the force applied to the self-inflating cuff is a compression force applied to the exterior surface of the first and second ends of the self-inflating cuff.

12. The method of claim 9, wherein the force applied to the self-inflating cuff is a tension force applied to the exterior surface of the first and second ends of the self-inflating cuff.

13. The method of claim 9, wherein the first compartment is adjacent to the second compartment within the self-inflating cuff.

14. The method of claim 9, wherein the first compartment is configured to rupture before the second compartment when the force is applied to the self-inflating cuff.

15. The method of claim 9, wherein the first compartment includes a first predetermined amount of the first substance and the second compartment includes a second predetermined amount of the second substance, wherein the first predetermined amount of the first substance and the second predetermined amount of the second substance are based on a volume of the self-inflating cuff.

16. The method of claim 9, wherein the self-inflating cuff comprise a resilient material such that, after the self-inflating cuff expands, the self-inflating cuff self-contracts at a predetermined rate until the self-inflating cuff returns to an unexpanded state.

17. An infusion system, comprising:
a fluid source;
tubing coupled with the fluid source; and
a self-inflating cuff, wherein:
the self-inflating cuff is configured to, responsive to a force applied to an exterior surface of the self-inflating cuff, i) initiate a chemical reaction caused by a first substance and a second substance within distinct compartments of the self-inflating cuff that are combined in response to the force, and ii) produce a reactionary force on an interior surface of the self-inflating cuff that causes the self-inflating cuff to expand; and
the self-inflating cuff expands such that the self-inflating cuff is configured to i) receive and couple with the fluid source and ii) pass the tubing through an opening of the self-inflating cuff such that the self-inflating cuff does not occlude the tubing.

18. The infusion system of claim 17, wherein the self-inflating cuff is configured to, after the reactionary force applied to the self-inflating cuff is removed at completion of the chemical reaction, self-contract and compress the fluid source such that a fluid flowing from the fluid source, when flowing, is accelerated.

19. The infusion system of claim 17, wherein the force applied to the exterior surface of the self-inflating cuff is a compression force.

20. The infusion system of claim 17, wherein the force applied to the exterior surface of the self-inflating cuff is a tension force.
